(19) United States
(12) Patent Application Publication
(10) Pub. No.: US 2026/0199020 A1
(43) Pub. Date: Jul. 16, 2026

(54) IMAGE-FREE SURGICAL NAVIGATION OF ANTERIOR CRUCIATE LIGAMENT (ACL) RECONSTRUCTION

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Cristóvão Jorge DA SILVA DUARTE E SOUSA, Coimbra (PT); Carolina DOS SANTOS RAPOSO, Coimbra (PT); João Pedro DE ALMEIDA BARRETO, Coimbra (PT); Rui Jorge MELO TEIXEIRA, Tondela (PT); Michel Gonçalves ALMEIDA ANTUNES, Coimbra (PT)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/563,233

(22) Filed: Mar. 11, 2026

Related U.S. Application Data
(63) Continuation of application No. PCT/US2024/046069, filed on Sep. 11, 2024.
(60) Provisional application No. 63/585,275, filed on Sep. 26, 2023.

(51) Int. Cl. A61B 34/10; A61B 34/20

(52) U.S. Cl. CPC A61B 34/10; A61B 34/20; A61B 2034/105; A61B 2034/2065

(57) ABSTRACT

A method includes receiving, by a computing device, a plurality of identified points corresponding to an intercondylar notch of a femur of a patient, a medial intercondylar arc of the femur, and a lateral intercondylar arc of the femur, determining, by the computing device and based on the plurality of points, a Blumensaat line, a medial contour of the medial intercondylar arc, and a lateral contour of the lateral intercondylar arc, determining, by the computing device, a sagittal plane for the femur based on the Blumensaat line, the medial contour of the medial intercondylar arc, and the lateral contour of the lateral intercondylar arc, and performing at least one function of a surgical procedure associated with the femur based on the determined sagittal plane.

### CROSS-REFERENCE TO RELATED APPLICATIONS

This application claims the benefit of PCT App. No. PCT/US2024/046069, filed Sep. 11, 2024, and U.S. Provisional App. 63/585,275, filed Sep. 26, 2023, titled “Image-Free Surgical Navigation of Anterior Cruciate Ligament (ACL) Reconstruction,” the entire contents of which are incorporated by reference herein.

### TECHNICAL FIELD

The present disclosure relates to preoperative and intraoperative surgical analysis and processing, and more particularly, to methodologies for anatomical reference frame (ARF) determinations of a bone and the automatic placement of a reference grid, such as the Bernard-Hertel's (BH) grid.

### BACKGROUND

The Anterior Cruciate Ligament (ACL) is one of the key ligaments that provide stability to the knee joint. Playing sports that involve sudden stops or changes in direction is one of the main causes for ACL injury, an example of which is its complete tear. For this reason, an ACL tear is a common medical condition with more than 200,000 annual cases per year in the United States alone. The standard way of treatment is arthroscopic reconstruction where the torn ligament is replaced by a tissue graft that is pulled into the knee joint through tunnels opened with a drill in both the femur and tibia. Opening these tunnels in an anatomically correct position ensures knee stability and patient satisfaction, though the current failure rates in primary ACL reconstructions range from 10-15%.

### SUMMARY

A method includes receiving, by a computing device, a plurality of identified points corresponding to an intercondylar notch of a femur of a patient, a medial intercondylar arc of the femur, and a lateral intercondylar arc of the femur, determining, by the computing device and based on the plurality of points, a Blumensaat line, a medial contour of the medial intercondylar arc, and a lateral contour of the lateral intercondylar arc, determining, by the computing device, a sagittal plane for the femur based on the Blumensaat line, the medial contour of the medial intercondylar arc, and the lateral contour of the lateral intercondylar arc, and performing at least one function of a surgical procedure associated with the femur based on the determined sagittal plane.

In accordance with one or more embodiments, the present disclosure provides a non-transitory computer-readable storage medium for carrying out the above-mentioned technical steps. The non-transitory computer-readable storage medium has tangibly stored thereon, or tangibly encoded thereon, computer readable instructions that, when executed by a device, cause at least one processor to perform a method for providing novel mechanisms for automatic placement of the BH grid and automatic determination of an ARF.

In accordance with one or more embodiments, a system is provided that comprises one or more computing devices and/or apparatus configured to provide functionality in accordance with such embodiments. In accordance with one or more embodiments, functionality is embodied in steps of a method performed by at least one computing device and/or apparatus. In accordance with one or more embodiments, program code (or program logic) executed by a processor(s) of a computing device to implement functionality in accordance with one or more such embodiments is embodied in, by and/or on a non-transitory computer-readable medium.

### BRIEF DESCRIPTION OF THE DRAWINGS

The features and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 is a block diagram of an example configuration within which the systems and methods disclosed herein could be implemented according to some embodiments of the present disclosure;

FIG. 2 is a block diagram illustrating components of an exemplary system according to some embodiments of the present disclosure;

FIG. 3 illustrates an exemplary data flow according to some embodiments of the present disclosure;

FIG. 4 depicts a non-limiting example embodiment of the disclosed technology according to some embodiments of the present disclosure;

FIG. 5 depicts a non-limiting example embodiment of the disclosed technology according to some embodiments of the present disclosure;

FIG. 6 depicts a non-limiting example embodiment of the disclosed technology according to some embodiments of the present disclosure;

FIG. 7 depicts a non-limiting example embodiment of the disclosed technology according to some embodiments of the present disclosure;

FIG. 8 depicts a non-limiting example embodiment of the disclosed technology according to some embodiments of the present disclosure;

FIG. 9 depicts a non-limiting example embodiment of the disclosed technology according to some embodiments of the present disclosure;

FIG. 10A shows an example 3D bone model including example locations identified during registration according to some embodiments of the present disclosure;

FIG. 10B shows an example Blumensaat's line L according to some embodiments of the present disclosure;

FIG. 10C shows example digitization of the medial intercondylar arc according to some embodiments of the present disclosure;

FIG. 10D shows example digitization of the lateral intercondylar arc according to some embodiments of the present disclosure;

FIG. 10E shows an example plane T′ obtained based on medial and lateral intercondylar contours according to some embodiments of the present disclosure;

FIGS. 10F and 10G illustrate mapping of relationships between world marker coordinates, XR plane coordinates, and anatomical coordinates according to some embodiments of the present disclosure;

FIG. 11A illustrates an example 3D bone model with three example landmarks acquired on a femur surface;

FIGS. 11B and 11C illustrate computation of the sagittal plane and BH grid according to some embodiments of the present disclosure;

FIG. 11D shows an example process or method for obtaining, intra-operatively, a BH grid from sparse 3D point/trajectory data according to some embodiments of the present disclosure; and

FIG. 12 is a block diagram illustrating a computing device showing an example of a device used in various embodiments of the present disclosure.

### DETAILED DESCRIPTION

The present disclosure will now be described more fully hereinafter with reference to the accompanying drawings, which form a part hereof, and which show, by way of non-limiting illustration, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as “up,” “down,” “bottom,” “top,” “front,” “rear,” “upper,” “lower,” “upwardly,” “downwardly,” and other orientational descriptors are intended to facilitate the description of the exemplary embodiments of the present disclosure, and are not intended to limit the structure of the exemplary embodiments of the present disclosure to any particular position or orientation. Terms of degree, such as “substantially” or “approximately,” are understood by those skilled in the art to refer to reasonable ranges around and including the given value and ranges outside the given value, for example, general tolerances associated with manufacturing, assembly, and use of the embodiments. The term “substantially,” when referring to a structure or characteristic, includes the characteristic that is mostly or entirely present in the characteristic or structure. As one example, numerical values that are described as “approximate” or “approximately” as used herein may refer to a value within +/−5% of the stated value.

For the purposes of this disclosure, a non-transitory computer readable medium (or computer-readable storage medium/media) stores computer data, which data can include computer program code (or computer-executable instructions) that is executable by a computer, in machine-readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, optical storage, cloud storage, magnetic storage devices, or any other physical or material medium which can be used to tangibly store the desired information or data or instructions and which can be accessed by a computer or processor.

For the purposes of this disclosure, the term “server” should be understood to refer to a service point that provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure, a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine-readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router (WR) mesh, or 2nd, 3rd, 4^{th} or 5^{th} generation (2G, 3G, 4G or 5G) cellular technology, mobile edge computing (MEC), Bluetooth, 802.11b/g/n, or the like. Network access technologies may enable wide area coverage for devices, such as client devices with varying degrees of mobility, for example. In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

For purposes of this disclosure, a client (or consumer or user) device, referred to as user equipment (UE)), may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device a Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

In some embodiments, as discussed below, the client device can also be, or can communicatively be coupled to, any type of known or to be known medical device (e.g., any type of Class I, II or III medical device), such as, but not limited to, a MRI machine, CT scanner, Electrocardiogram (ECG or EKG) device, photopletismograph (PPG), Doppler and transmit-time flow meter, laser Doppler, an endoscopic device neuromodulation device, a neurostimulation device, and the like, or some combination thereof.

The position and orientation of a femoral tunnel for ACL reconstruction significantly impacts the success of the surgery, motivating the need for a pre-operative plan for properly defining the best femoral tunnel. In order to determine the anatomically correct position of the femoral tunnel, some surgeons rely on specific anatomical landmarks. However, these landmarks may not be reliable and may even not exist in some patients. In order to obtain more accurate femoral tunnel positions, a Bernard-Hertel's (BH) grid can be utilized. The BH grid can be utilized for proposing ACL reconstruction techniques and for assessing tunnel placement after ACL reconstruction.

By way of background, BH grids involve a quadrant method for determining the location of the femoral insertion. Using a lateral radiograph, the Blumensaat's line can be identified and two other lines perpendicular to that one can be drawn such that the lines go through the shallow and the deep borders of the lateral femoral condyle. A fourth line to be drawn is parallel to Blumensaat's line and is tangent to the inferior border of the condyles. The obtained BH grid consists of a normalized reference frame that is independent of knee size, shape and distance at which the X-ray was acquired. Coordinates on this reference frame are given as percentages along Blumensaat's line and the perpendicular direction.

In some conventional examples, the BH grid system is applied to pre-operative images (e.g., X-ray, MRI, or CT images), such as lateral radiographs of the knee. However, image quality and a direction of the X-ray tube with respect to the patient can influence the accuracy of tunnel location measurement. A common challenge of imaging methodologies is that the placement of BH grid, whether using radiographs or computerized tomography (CT) imaging, is a manual process subject to observer variability. For example, despite an apparent improved inter-observer agreement obtained with CT scans (when compared to radiographs), the variability in measuring the femoral tunnel location is still non-negligible, making such techniques unreliable.

Example surgical navigation systems and methods may include a pre-operative phase and an intra-operative phase. In the pre-operative phase, the patient is required to get an MRI or CT scan of the knee, which is segmented (manually or automatically) to obtain a 3D model of the femur. The 3D model is used in a surgical planning phase to place the BH grid system, which is then used in the intra-operative phase. In the intra-operative phase, the 3D model is registered with intra-operative anatomical data that is obtained by the surgeon, which facilitates pre-operative surgical planning by the surgeon, provides support during the intra-operative surgical planning, and implements augmented and virtual reality capabilities.

These example surgical navigation systems and methods are comprised of image-based navigation since pre-operative imaging is performed. As discussed above, image-based navigation requires a 3D model that is computed from a pre-operative medical scan (X-ray, MRI, CT, etc.) after segmentation of the anatomy of interest. The segmentation can be performed manually or automatically through artificial intelligence (AI) based models. The BH grid system is subsequently placed (e.g., manually by a surgeon, using pre-operative planning software or other 3D techniques, etc.) during the pre-operative surgical planning.

Surgical navigation systems and methods according to the principles of the present disclosure are configured to perform video-based navigation for knee arthroscopy (e.g., navigation of the femoral ACL tunnel) using image-free navigation techniques. The systems and methods of the present disclosure include placement of the BH grid system in the patient's anatomy without requiring a pre-operative medical scan or other pre-operative data. In addition to allowing planning of the ACL femoral tunnel, the placement of the BH grid in accordance with the principles of the present disclosure facilitates determination of an initial alignment between the patient's anatomy and a statistical shape model (SSM), greatly benefiting bone morphing.

As described below in more detail, the systems and methods of the present disclosure achieve automatic placement of the BH grid system without requiring a medical scan (e.g., using sparse 3D data acquired intra-operatively, in contrast to existing solutions that require a medical scan to obtain a 3D anatomical model). In an example, a complete model of the femur bone can be obtained from a sparse set of 3D points acquired intra-operatively. For example, an initial alignment between the sparse set of 3D points and the SSM is obtained based on four corner points of the BH grid.

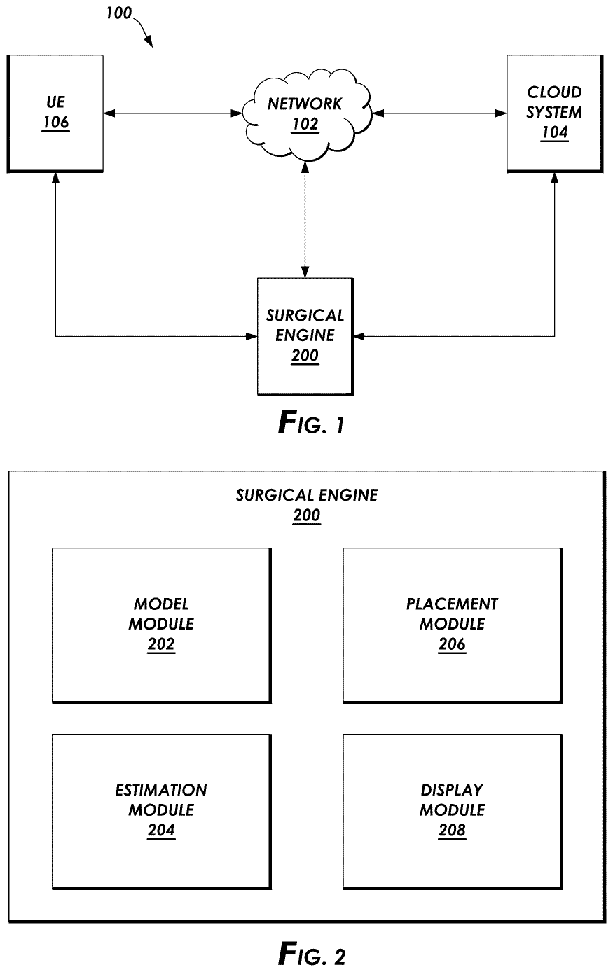

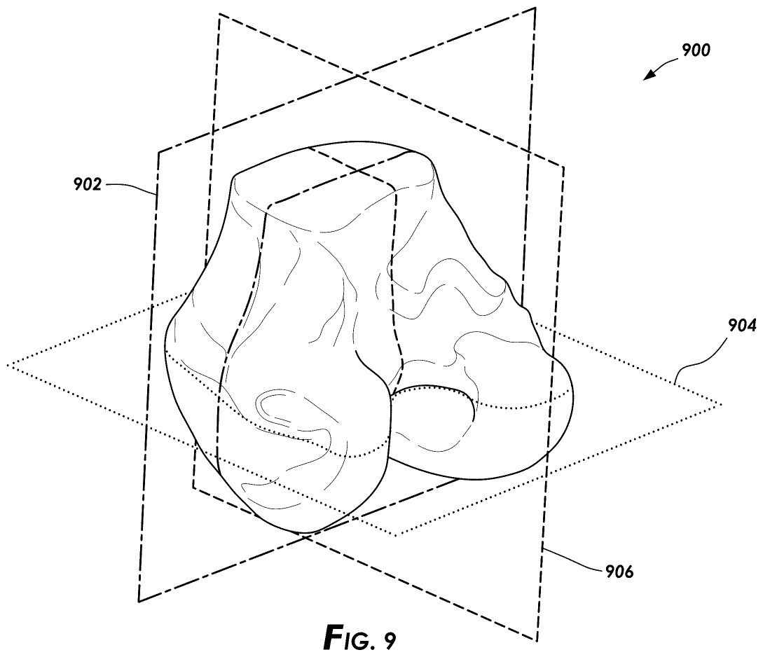

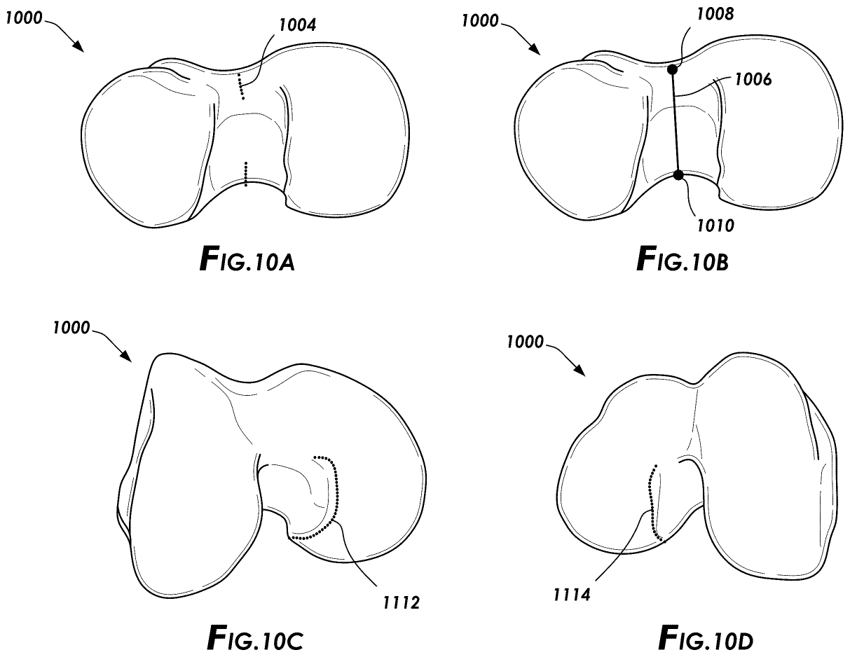

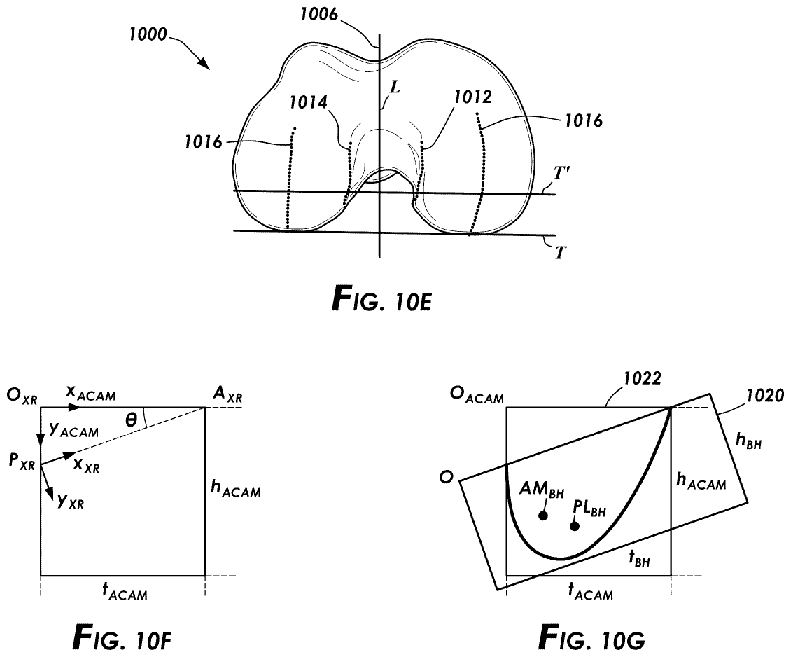

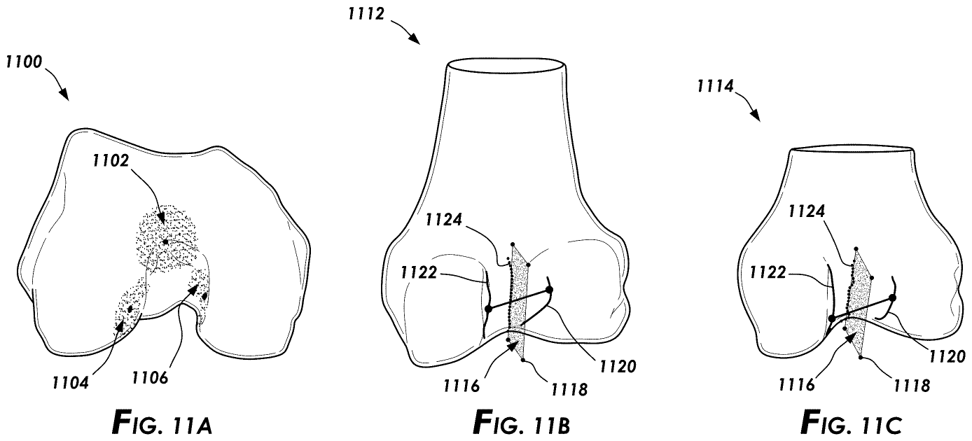

FIG. 1 shows an example system (or framework) 100 configured to implement one or more functions of the surgical navigation systems and methods of the present disclosure. The system 100 includes a UE 106 (e.g., a client device), a network 102, a cloud system 104, and a surgical engine 200. The UE 106 can be any type of device, such as, but not limited to, a mobile phone, tablet, laptop, personal computer, sensor, Internet of Things (IoT) device, autonomous machine, and any other device equipped with a cellular, wireless, or wired transceiver. In some embodiments, as discussed above, the UE 106 can also be a medical device, or another device that is communicatively coupled to a medical device, that enables reception of readings from sensors of the medical device. For example, in some embodiments, the UE 106 can be a user's smartphone (or office/hospital equipment, for example) that is connected via WiFi, Bluetooth Low Energy (BLE) or NFC, for example, to a peripheral neuromodulation device. Thus, in some embodiments, the UE 106 can be configured to receive data from sensors associated with a medical device, as discussed in more detail below. Further discussion of the UE 106 is provided below at least in reference to FIGS. 10A and 10B.

Network 102 can be any type of network, such as, but not limited to, a wireless network, cellular network, the Internet, a local-area network, or a wide-area network. As discussed herein, network 102 can facilitate connectivity of the components of system 100, as illustrated in FIG. 1.

The cloud system 104 can be any type of cloud operating platform and/or network based system upon which applications, operations, and/or other forms of network resources can be located. For example, system 104 can correspond to a service provider, network provider and/or medical provider from where services and/or applications can be accessed, sourced or executed from. In some embodiments, the cloud system 104 can include a server(s) and/or a database of information that is accessible over network 102. In some embodiments, a database (not shown) of system 104 can store a dataset of data and metadata associated with local and/or network information related to a user(s) of the UE 106, patients and the UE 106, and the services and applications provided by cloud system 104 and/or surgical engine 200.

The surgical engine 200, as discussed below in more detail, includes components configured to perform image-free, automatic placement of a reference grid for a bone, such as a BH grid system. Embodiments of how engine 200 operates and functions, and the capabilities it includes and executes, among other functions, are discussed below in more detail.

According to some embodiments, surgical engine 200 can be a special purpose machine or processor and could be hosted by a device on network 102, within cloud system 104 and/or on UE 106. In some embodiments, engine 200 can be hosted by a peripheral device connected to the UE 106 (e.g., a medical device, as discussed above).

According to some embodiments, surgical engine 200 can function as an application provided by cloud system 104. In some embodiments, engine 200 can function as an application installed on the UE 106. In some embodiments, such application can be a web-based application accessed by the UE 106 over network 102 from cloud system 104 (e.g., as indicated by the connection between network 102 and engine 200, and/or the dashed line between the UE 106 and engine 200 in FIG. 1). In some embodiments, engine 200 can be configured and/or installed as an augmenting script, program or application (e.g., a plug-in or extension) to another application or program provided by cloud system 104 and/or executing on the UE 106.

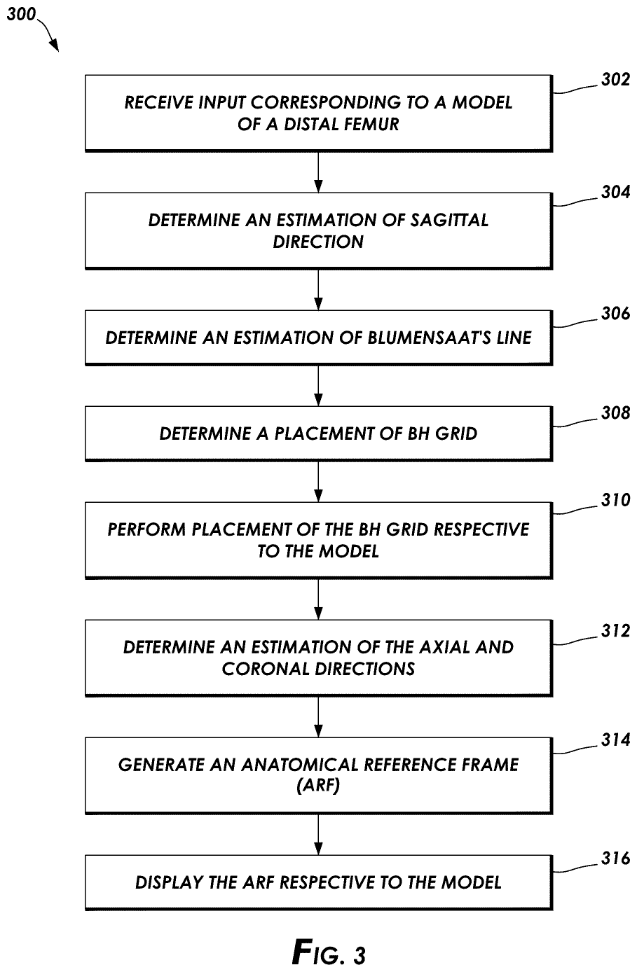

As illustrated in FIG. 2, according to some embodiments, surgical engine 200 includes a model module 202, an estimation module 204, a placement module 206, and a display module 208. It should be understood that the engine(s) and modules discussed herein are non-exhaustive, as additional or fewer engines and/or modules (or sub-modules) may be applicable to the embodiments of the systems and methods discussed. More detail of the operations, configurations and functionalities of engine 200 and each of its modules, and their role within embodiments of the present disclosure will be discussed below.

FIG. 3 shows an example Process 300 for determining an ARF of a bone pursuant to the automatic placement of a BH grid by using, as input, an image of the bone (e.g., a 3D model). In some examples, the sagittal direction is determined through alignment of the medial and lateral condyles and a radiographic view of the distal femur is generated for detecting the intercondylar contour. A BH grid is then obtained as the rectangle that is tangent to this contour and encloses the radiographic view of one or both condyles. The axial direction is determined automatically by using the estimated sagittal plane for retrieving a sagittal view of the bone shaft, from which circles can be extracted. By robustly joining the center of these circles, the axial direction is obtained. The cross-product between the sagittal and axial directions yields the coronal one, providing the complete ARF. This technique is applicable to different femur shapes and sizes and improves reliability, accuracy, and ease of implementation.

According to some examples, Step 302 of Process 300 can be performed by model module 202 of surgical engine 200; Steps 304-306 and 310-312 can be performed by estimation module 204; Steps 308 and 314 can be performed by placement module 206; and Step 316 can be performed by display module 208.

Process 300 begins with Step 302 where engine 200 receives input that identifies a 3D model of a distal femur. According to some embodiments, the identification of the 3D model can be based on, but not limited to, a request to generate a 3D model, the search for and retrieval of a 3D model, and/or an upload and/or download of a 3D model. In some embodiments, the input can be in the form an image, message, multi-media item, and/or any other type of known or to be known format for engine 200 to receive and process for display a digital content corresponding to a model (e.g., 3D model) of a patient's bone, and particularly the distal femur.

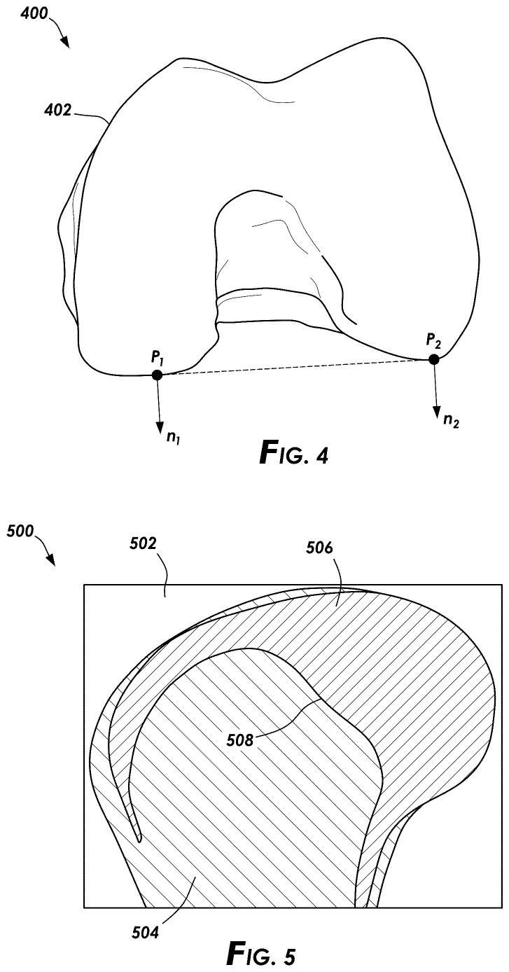

In Step 304, engine 200 performs an estimation of the sagittal direction based on the received input from Step 302. According to some embodiments, Step 304 involves engine 200 obtaining the sagittal direction, such as by estimating the sagittal direction of the bone based on pairs of points whose normal vectors are orthogonal to the vector joining them. According to some embodiments, this is illustrated in example 400 in FIG. 4, where the example distal femur 402 has identified thereon pairs of points P₁ and P₂ and normal vectors N₁ and N₂, respectively.

According to some embodiments, Step 304 can involve, based on the input of the 3D model, computing a normal for every point (or at least a set of points on the bone). According to some embodiments, Step 304 can be restricted in the search domain when searching for points by finding a region of interest (ROI) that contains the condyle surface, where only points on that ROI (instead of the full 3D model) are considered. In some embodiments, ROI can be found by registering the 3D model with a template model, by making use of a statistical shape model (SSM), through 3D curvature analysis, using deep learning frameworks, or some combination thereof.

Engine 200 can then analyze the computed normals and determine a pair of points (e.g., P₁ and P₂) for which the corresponding normals (N₁ and N₂) are parallel, and vector “v” joining P₁ and P₂ is orthogonal to N₁ and N₂, as illustrated in FIG. 4.

Engine 200 can then determine the sagittal direction based on the hypothesis: v=P₂−P₁. According to some embodiments, the vector v joining each selected pair of points consists of a hypothesis for the sagittal direction. Based on such sagittal direction hypotheses, an estimation of the sagittal direction can be determined. In some embodiments, the hypotheses can be represented as 3D points and then clustered, whereby a median value of the cluster can be computed. In some other embodiments, Random Sample Consensus (RANSAC), or other robust estimation models (e.g., Hough transform) can be applied to the set of hypotheses to estimate the sagittal direction.

According to some embodiments, Step 304 can further involve determining a lateral-to-medial orientation to the sagittal direction by identifying lateral and medial condyles (e.g., respective to P₁ and P₂).

In some embodiments, the estimation of the sagittal direction of Step 304 can further involve refining the sagittal direction by generating a simulated radiographic view of the femur or a two-dimensional (2D) intersection map (as discussed below in relation to at least Step 306 and FIG. 5), and adjusting the outer border of the condyles such that they overlap. Such generated views/maps can be generated by considering orthographic or perspective projections.

Process 300 proceeds from Step 304 to Step 306 where, having determined the estimation of the sagittal direction (Step 304), engine 200 performs an estimation of Blumensaat's line. According to some embodiments, engine 200 accesses the 3D model of the femur, and builds a 2D projection of the number of intersections of projection rays with the 3D model (referred to as the “intersection map”). A non-limiting example of such mapping is provided in FIG. 5, where intersection map 500 includes regions 502, 504, and 506, and a curve 508. As depicted in FIG. 5, region 502 corresponds to zero (0) intersections, region 504 corresponds to regions with two (2) intersections, and region 506 corresponds to regions with four (4) intersections. According to some embodiments, intersection map 500 enables the identification of a curve 508 (e.g., curve “C”). Curve 508 corresponds to the contour of the intercondylar region, which can be determined by performing an edge detection analysis (or computation) and retrieving the curve between regions with intersections 2 and 4 (e.g., between regions 504 and 506, respectively, in FIG. 5).

According to some embodiments, the determination at Step 306 of Blumensaat's line can involve finding the line that is tangent to the curve 508 in the largest number of points that does not intersect it. In other words, regardless of shape, Blumensaat's line is tangent to curve 508 but does not intersect curve 508 despite being tangential. This step is not dependent on particular curvature patterns.

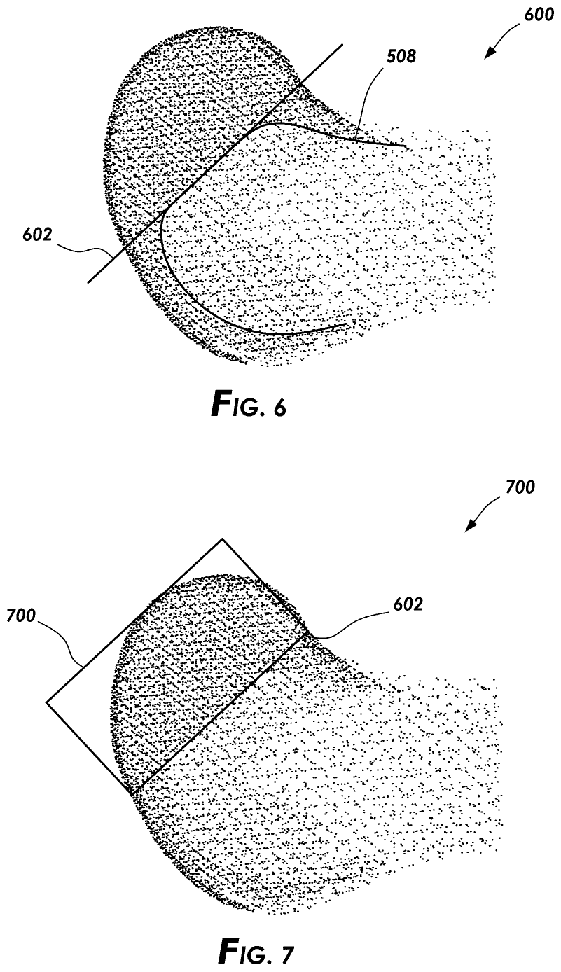

Turning to FIG. 6, illustrated in 2D projection 600 of a distal femur from a side view, where Blumensaat's line 602 is shown in relation to curve 508. Thus, as depicted in FIG. 6 and described herein in relation to Step 306, Blumensaat's line is tangent to curve 508 in the largest number of points and does not intersect curve 508 elsewhere. According to some embodiments, Blumensaat's line can be determined based on the use of a template model/SSM, 2D curvature analysis, deep learning scheme, voting scheme, clustering, and/or any other type of known or to be known heuristics.

In some embodiments, in situations where curve 408 is a hill type, Blumensaat's line can intersect some region of the intercondylar contour and be tangent to it only in a specified location (e.g. near the intercondylar notch). In some embodiments, Blumensaat's line can be further based on a backprojection of the 2D points of intersection map 500 to points on the 3D model. In some embodiments, the backprojection can be based on a sectioning plane defined by the sagittal direction (from Step 304). Thus, according to some embodiments, 3D points in the 3D model can be retrieved/determined by backprojecting the intercondylar contour/Blumensaat's line onto the 3D model. Having such points, an appropriate sectioning plane of the model can be obtained based on, for example, the plane with sagittal direction that contains such points. In some embodiments, such sectioning plane can then be used when determining the axial direction through circle fitting in the shaft, as discussed below.

Process 300 then proceeds from Step 306 to Step 308 where engine 200 determines a placement of a BH grid. Engine 200 determines the placement (and other characteristics, such as, for example, size, proportions and dimensions) of the BH grid on the estimates for the sagittal direction (from Step 304) and Blumensaat's line (from Step 306).

According to some embodiments, Step 308 involves engine 200 placing the BH grid such that it encloses the condyles when depicted in a lateral view of the distal femur. According to some embodiments, edge detection is applied to an intersection map (e.g., map 500 from FIG. 5), where the edge corresponding to the curve enclosing the region of zero intersections is retrieved (which corresponds to the sagittal contour of the condyles). In other words, engine 200 utilizes a 2D projection of the number of intersections of projection rays along the sagittal direction with the bone model when considering an orthographic projection. Then, Blumensaat's line (line 602 from FIG. 6) is intersected with the obtained contour, yielding the long edge of BH grid. Finally, the line parallel to Blumensaat's line that is tangent to the contour is obtained, and the distance between both lines is the length of the short edge (width) of BH grid. An example of this is depicted in FIG. 7, wherein a 2D projection 700 is depicted, which includes Blumensaat's line 602 and BH grid 702. According to some embodiments, projection 700 can be a radiographic view or an intersection map obtained from either an orthographic or perspective projection. In some embodiments, if the location of the lateral condyle is known, edge detection of Step 306 can be accomplished by firstly sectioning the model sagittally and considering only the lateral condyle for building the intersection map.

In some embodiments, Step 308 can further involve backprojecting the BH grid from the 2D model to the 3D model. In such embodiments, which is realized in Step 310, engine 200 can perform the backprojection so that the BH grid is displayed as part of or as an overlay of the 3D model. In some embodiments, the backprojection can be based on a sectioning plane defined by the sagittal direction and Blumensaat's line.

Process 300 then proceeds from Step 310 to Step 312 where engine 200 an estimation of the axial and coronal directions (e.g., remaining anatomical directions) are determined. As discussed herein, the axial and coronal directions are utilized to determine ARF.

According to some embodiments, Step 312 can include a set of sub-steps. A first sub-step involves engine 200 obtaining a sagittal view of the bone from which the contours of the shaft are retrieved. In some embodiments, the sagittal view can be an orthographic projection of the entire or sectioned femur model, or obtained from intersection of the sectioning plane with the model. In some embodiments, the sagittal view can be an intersection map (as described above in relation to FIG. 5), where the contour of the shaft can be obtained based on the transition between regions of 0 and 2 intersections (e.g., region 502 and 504, respectively).

In the next sub-step, engine 200 performs a search for the circles that are tangent (e.g., simultaneously tangent) to the shaft contour in two points, where the line that joins the centers of the obtained circles provides an estimate for the axial direction. In some embodiments, in cases where the anterior and posterior cortices of the femur are known, the search can be restricted by considering only pairs containing one point from each cortex. According to some embodiments, the axial direction can be alternatively determined based on a determined relationship between a fixed angle (at a predetermined value) with respect to Blumensaat's line. In some embodiments, the axial direction can be alternatively determined based on a cylinder fitting methodology utilizing dimensions and values of the shaft region.

Engine 200 can then determine the coronal direction via the cross-product between the sagittal and axial directions.

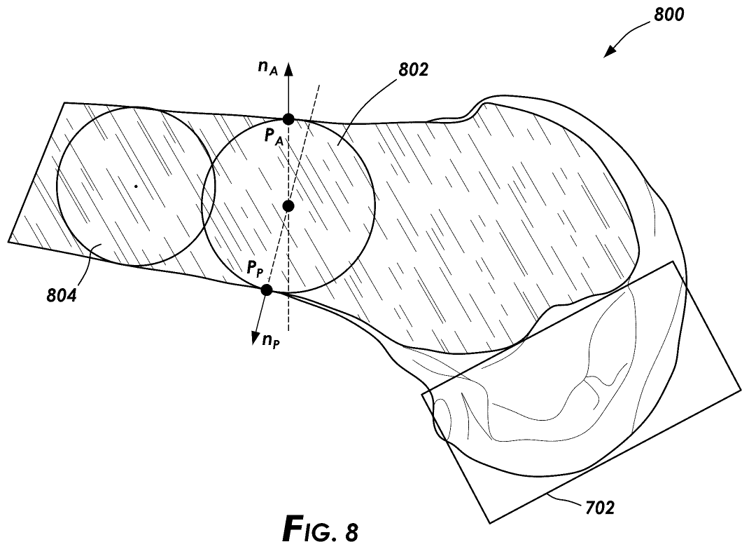

FIG. 8 depicts an example of a femur model 800, where circles 802 and 804 are depicted. Circles 802 and 804, as discussed above, are tangent to the shaft contour and can be obtained as follows. First, the normal at each point in the shaft contour is computed. Then, all pairs of contour points are generated and the lines going through them that are parallel to the respective normal vectors are intersected. According to FIG. 8, the line with direction n_{A} that contains point P_{A} intersects with the line with direction np that contains point P_{P} on the center of circle 802, which belongs to the axial direction. By joining all intersection points that are equidistant from the considered points, which correspond to centers of circles tangent to the shaft contour, the axial direction is obtained.

According to some embodiments, the step of joining the points can be performed using any known or to be known technique, algorithm or mechanism, such as, but not limited to, standard or robust line fitting, clustering schemes, Hough transforms, and/or any other known or to be known technique for estimating and determining lines (and their distances/length) from sets of points.

Turning back to Process 300, Process 300 proceeds from Step 312 to Step 314 where engine 200 generates an ARF for the distal femur. As illustrated in FIG. 9, an example of a generated ARF 900 is depicted, which includes sagittal direction 906, axial direction 904 and coronal direction 902.

In Step 316, the generated ARF can be displayed as an overlay or part of the 3D model, which can be used for an ACL procedure, as discussed above. In some embodiments, the information related to the ARF, directions, BH grid and Blumensaat's line can be stored and utilized for subsequent ARF projections.

As such, based at least on the discussion above, the techniques described in FIGS. 3-9 function without requiring alignment with a template model, nor initialization of the sagittal direction. These techniques can be performed without an entire femur model, and does not depend on the curvature pattern of the intercondylar contour. This, among other benefits, enables these techniques to be applicable to a wider variety of input models and different types of morphologies, and evidences a system that works in a more computationally efficient and accurate manner, while not being prone to suffer from local minima issues.

The techniques for placing the BH grid as described above assume a lateral radiographic view of the distal femur, from which other information can obtained. The lateral radiographic view can be generated by orthographic projection of the femur along the sagittal direction.

Systems and methods according to the principles of the present disclosure are configured to determine a correct placement of a BH grid directly with respect to patient anatomy, instead of with respect to a bone model of the patient anatomy generated using pre-operative (e.g., MRI) images. By directly determining a correct placement of a BH grid, planning of an ACL femoral tunnel can be done without creating such a bone model, and therefore further without requiring intraoperative registration between the bone model and the patient anatomy. Furthermore, direct BH grid placement can be useful for determining an initial alignment between the patient anatomy and a statistical shape model (SSM) to be used for bone morphing.

A method of determining a correct placement of a BH grid is preceded by a process of determining a Blumensaat's line and determining the sagittal plane (i.e., of the femur) in which the Blumensaat's line sits. The method starts with the surgeon collecting 3D points along the roof of the intercondylar notch in order to begin to define Blumensaat's line. Blumensaat's line is the line that is tangent to the roof of the intercondylar notch. Collecting the 3D points may be done by registering the relative 3D positions of a tracked tool fiducial and a tracked bone fiducial in arthroscopic images as a surgeon touches a tip of the tool to a number of locations along the anterior and posterior sections of the roof of the intercondylar notch.

FIG. 10A shows an example 3D bone model 1000 including example locations 1004, such as digitized points, which may be identified by the surgeon. These points are shown on the bone model 1000 simply as an example of the locations of these points relative to a femur (i.e., an image such as a bone model is not actually obtained or used). Rather, these points are obtained directly from patient anatomy. These locations 1004 along anterior and posterior sections of an intercondylar notch are collected as a set S={X₁, X₂, . . . , X_{N}}, with Xᵢ being a 3D point and N≥2 being the number of 3D points in S.

Blumensaat's line L is a line fitted to S using a line fitting algorithm. As shown in FIG. 10B, Blumensaat's line L is shown at 1006 having end points 1008 and 1010 that the surgeon has defined as respective 3D locations. Blumensaat's line L can be defined as:

$$L = ( \begin{matrix}u \\ v\end{matrix} )$$

$$where :$$

$$u = \frac{A - P}{‖ A - P ‖}$$

$$and :$$

$$v = \frac{A \times P}{‖ A \times P ‖} ,$$

where A corresponds to an anterior endpoint (e.g., as shown at 1008) and P corresponds to a posterior endpoint (e.g., as shown at 1010).

Blumensaat's line L extends along the sagittal plane Π, but the sagittal plane is but one plane in a pencil of planes Π(λ) defined by Blumensaat's line L. Therefore, further steps are required in order to enforce constraints for determining the value of λ (i.e., to single out the sagittal plane Π from the pencil of planes Π(λ)).

An additional constraint for this purpose is obtained by assuming that “paths of contact” of the condyles of the femur with the tibia during knee joint rotation are two parallel 3D curves. Based on this assumption, a vector joining corresponding points in both curves, along with respective normal of the points, can be used to define a plane T that, in turn, can be used to define the sagittal direction.

The condyles themselves are not captured in arthroscopic images as they are outside of the field of view. However, an assumption is made that the intercondylar contours, which themselves can be within the field of view, run parallel to the contours of the condyles. Therefore, with a view to obtaining a plane T′ that runs parallel to T, a surgeon may collect 3D points along the medial and lateral intercondylar arcs to define medial (M) and lateral (L) intercondylar contours having sets of a number K of 3D points X:

$${C}_{L} = \{ {X}_{L}^{1} , {X}_{L}^{2} , … , {X}_{L}^{{K}_{L}} \}$$

$${C}_{M} = \{ {X}_{M}^{1} , {X}_{M}^{2} , … , {X}_{M}^{{K}_{M}} \}$$

Where C_{L} (or C_{L}) defines the lateral contour, C_{M} (or C_{M}) defines the medial contour,

$${X}_{L / M}^{i}$$

is a 3D point in the contour, and K_{L} and K_{M} are the number of 3D points in the lateral and medial contours, respectively.

FIG. 10C shows example digitization of the medial intercondylar arc at 1112. FIG. 10D shows example digitization of the lateral intercondylar arc at 1114.

FIG. 10E shows another view of the intercondylar arcs 1112 and 1114 and condyle curves 1016. The condyle curves 1016 typically are not accessible during arthroscopy (i.e., intra-operatively) because a field-of-view of the arthroscope is limited to the intercondylar region. It can be assumed that the intercondylar contours (as shown at 1112 and 1114) are generally parallel to the condyle curves 1016 in the region of interest. It can further be assumed that “paths of contact” between the condyles and the tibia during flexion of the knee joint are two parallel curves, represented as the condyle curves 1016. Corresponding points of contact along these paths, together with the normals of (i.e., lines normal to) the points of contact, define a plane T. A similar assumption can be made for points of the intercondylar arcs 1112 and 1114, which also define a plane T′ parallel to the plane T. More specifically, an assumption can be made that the intercondylar contours are parallel to the curves 1016 and therefore a plane T′ that is parallel to T can be determined.

Accordingly, with the lateral and medial intercondylar contours CL, CM having been established, a plane T′ may be computed by first applying a 3D tangent estimator to each contour to compute a tangent vector

$${t}_{L / M}^{i}$$

for each 3D point captured on the lateral and medial intercondylar contours CL, CM:

$${T}_{L} = \{ {t}_{L}^{1} , {t}_{L}^{2} , ... , {t}_{L}^{{K}_{L}} \}$$

$${T}_{M} = \{ {t}_{M}^{1} , {t}_{M}^{2} , ... , {t}_{M}^{{K}_{M}} \}$$

With the tangents having been estimated, a search process locates two (2) corresponding points

$${X}_{L}^{i} and {X}_{M}^{j}$$

on each of the lateral and medial intercondylar contours CL, CM that: (1) have parallel tangents

$${t}_{L}^{i} and {t}_{M}^{i}$$

and (2) form a line

$${X}_{L}^{i} - {X}_{M}^{j}$$

that is orthogonal to the direction u of the Blumensaat's line L established previously. With the two corresponding points

$${X}_{L}^{i} and {X}_{M}^{j}$$

having been found using the search process, a normal d to the plane T′ may be obtained by solving the equation:

$$( \begin{matrix}{( u )}^{T} \\ {( {X}_{L}^{i} - {X}_{M}^{j} )}^{T} \\ {( {t}_{L}^{i} )}^{T} \\ {( {t}_{M}^{j} )}^{T}\end{matrix} ) d = 0 ,$$

where superscript T denotes the transpose operator.

With the normal d to plane T′ having been established, λ for the sagittal plane can be computed using the following equation:

$$\prod ( λ ) ( \begin{matrix}d \\ 0\end{matrix} ) = 0$$

The sagittal plane parameters are given by substituting the estimated value of λ (as computed above) in the previously computed pencil of planes Π(λ) on which Blumensaat's line lies.

In some examples, the search process may be implemented for more than one pair of points, such as by implementing an optimization strategy using multiple pairs. In some examples, K_{L} and K_{M} may be very large. Accordingly, using a random sampling strategy or a coarse-to-fine strategy for the search might accelerate the search process. In other examples, down-sampling the contour points may be used to accelerate the search process.

In some examples, the tangents may be noisy, which can cause difficulties in finding a pair where both tangents are accurate. Accordingly, in some examples, a scoring function that takes left and right contour points into consideration separately may be used.

Referring now to FIGS. 10F and 10G, with Blumensaat's line A-P having been established, and with the sagittal plane containing Blumensaat's line A-P having been established, world marker coordinates are mapped to XR plane coordinates, and then XR plane coordinates are mapped to anatomical coordinates. Following this, a BH grid representation in the anatomical coordinates can be computed. These aspects are explained below with the assumption that the marker/fiducial is always located on the interior wall of the lateral condyle, and that the XR view is to be seen from the right side of the patient (i.e., the XR view is the lateral view in the case of the right knee and is the medial view in the case of the left knee). It will be appreciated that, if the marker/fiducial is placed elsewhere, the following calculations would be adapted accordingly.

To first map the world marker coordinates to the XR plane coordinates, with reference to FIG. 10F, a 3×4 matrix P_{XR} that maps points X in 3D world coordinates into points X_{XR} in 2D X-Ray (XR) coordinates is to be determined according to:

$${X}_{XR} ∼ {P}_{XR} X$$

In order to obtain the matrix P_{XR} it may be considered that n is the normal of the sagittal plane Π defined previously, the points A and P the anterior and posterior limits of Blumensaat's line L, and the corresponding direction of Blumensaat's line

$$u = \frac{( A - P )}{‖ A - P ‖} .$$

Based on this, P_{XR} may be calculated as follows:

$${P}_{XR} = ( \begin{matrix}1000 \\ 0100 \\ 0001\end{matrix} ) ( \begin{matrix}{R}^{T} & - {R}^{T} P \\ 0 & 1\end{matrix} )$$

with R=(u n×u n) and P is the origin of the XR coordinate system.

To thereafter map XR coordinates to the anatomical coordinate system (anatomical coordinates axes measures, or ACAM), it may be considered that, with Blumensaat's line having an inclination θ relative to the horizontal direction in case of 90° knee flexion, θ may be set at approximately 33 degrees. In other examples, θ may be set to different values.

Based on the above, and assuming I as the distance between A and P, the transformation from XR coordinates to ACAM coordinates may be given by:

$${X}_{ACAM} = {P}_{ACAM} {X}_{XR}$$

$$where {P}_{ACAM} = ( \begin{matrix}cos ( θ ) & sin ( θ ) & 0 \\ - sin ( θ ) & cos ( θ ) & l sin ( θ ) \\ 0 & 0 & 1\end{matrix} ) .$$

Because t_{ACAM}=lcos (θ), to obtain h_{ACAM} all contour points are mapped into ACAM and the contour point having the maximum y_{ACAM} coordinate is selected. That is, h_{ACAM}=max (y_{ACAM}). In another example, only contour points corresponding to the lateral contour may be considered as described above.

The BH grid representation is related to the anatomical coordinate system by a mapping, represented in FIG. 10G at 1020 as coordinates in BH grid space, and at 1022 as coordinates in ACAM space. A location AM is the center of the anteromedial tunnel, and a location PL is the center of the posterolateral tunnel when considering a double-bundle ACL reconstruction technique.

The relationships between the BH grid and the ACAM as described above may be based on anatomical femur statistics. Example matrix calculations may be used to establish relative locations of anteromedial and posterolateral tunnels' entry points (AM, PL) with respect to the BH grid, and to establish relative locations of AM and PL with respect to the ACAM coordinate system. The correspondences between these four points can then be used to establish a transformation between the BH grid and ACAM coordinate systems.

With the transformation between the BH grid and the ACAM coordinate systems having been established, a chain of transformations can be formed that enable a transformation of a 3D point X in the world coordinate system (as established based on the marker in the arthroscopic field of view) to a point XBH in the BH grid coordinate system.

In an example, various experimental results suggest that the anatomic posterior-to-anterior direction of the anteromedial and posterolateral tunnels' entry points were located at 23.1%±6.1% and 15.3%±4.8%, respectively (100% minus these values in relation to h_{ACAM}). The proximal-to-distal locations were at 28.2%±5.4% and 58.1%±7.1%, respectively. With the BH quadrant method, anteromedial and posterolateral tunnels were measured at 21.7%±2.5% and 35.1%±3.5%, respectively from the proximal condylar surface parallel to the Blumensaat line (which corresponds to t_{BH}), and at 33.2%±5.6% and 55.3%±5.3% from the notch roof perpendicular to the Blumensaat line (which corresponds to h_{BH}). While these values are used in the below example, the principles of the present disclosure are not restricted to these values and other values or measurements may be used.

In accordance with these example values and in view of the principles described above with respect to FIG. 10G:

$${AM}_{BH} = ( \begin{matrix}{t}_{BH} & 0 & 0 \\ 0 & {h}_{BH} & 0 \\ 0 & 0 & 1\end{matrix} ) ( \begin{matrix}0.217 \pm 0.025 \\ 0.332 \pm 0.056 \\ 1\end{matrix} )$$

$${PL}_{BH} = ( \begin{matrix}{t}_{BH} & 0 & 0 \\ 0 & {h}_{BH} & 0 \\ 0 & 0 & 1\end{matrix} ) ( \begin{matrix}0.351 \pm 0.035 \\ 0.553 \pm 0.053 \\ 1\end{matrix} )$$

$${AM}_{ACAM} = ( \begin{matrix}{t}_{ACAM} & 0 & 0 \\ 0 & {h}_{ACAM} & 0 \\ 0 & 0 & 1\end{matrix} ) ( \begin{matrix}0.282 \pm 0.054 \\ 0.769 \pm 0.061 \\ 1\end{matrix} )$$

$${PL}_{ACAM} = ( \begin{matrix}{t}_{ACAM} & 0 & 0 \\ 0 & {h}_{ACAM} & 0 \\ 0 & 0 & 1\end{matrix} ) ( \begin{matrix}0.581 \pm 0.071 \\ 0.847 \pm 0.048 \\ 1\end{matrix} )$$

A transformation from ACAM to the BH reference frame can be obtained using the correspondences (AM_{BH}, AM_{ACAM}) and (PL_{BH}, PL_{ACAM}) in accordance with:

$${X}_{BH} = {B}_{BH} {D}_{ACAM} {X}_{ACAM}$$

$$with$$

$${D}_{ACAM} = ( \begin{matrix}{t}_{ACAM} & 0 & 0 \\ 0 & {h}_{ACAM} & 0 \\ 0 & 0 & 1\end{matrix} ) ,$$

$${B}_{BH} = ( \begin{matrix}1 / {t}_{BH} & 0 & 0 \\ 0 & 1 / {h}_{BH} & 0 \\ 0 & 0 & 1\end{matrix} ) ( \begin{matrix}cos ( θ ) & - sin ( θ ) & a \\ sin ( θ ) & cos ( θ ) & b \\ 0 & 0 & 1\end{matrix} ) ,$$

where the unknowns are t_{BH}, h_{BH}, a, and b. This system can be solved considering that B_{BH} ⁻¹X_{BH}−D_{ACAM}X_{ACAM}=0. From this optimization, the values of t_{BH}, h_{BH}, a, and b with respect to t_{ACAM} and h_{ACAM} can be obtained.

The transformation that maps points in X 3D world coordinates to XBH BH grid coordinates is given by the three partial transformations described above. In other words, the above calculations enable a transformation of a 3D point X in the world coordinate system (as established based on the marker in the arthroscopic field of view) to a point XBH in the BH grid coordinate system according to:

$${X}_{BH} = {P}_{BH} {P}_{ACAM} {P}_{XR} X \qquad ( Equation 1 )$$

$$where {P}_{BH} = [ \begin{matrix}cos ( θ ) & - sin ( θ ) & 0 & a \\ sin ( θ ) & cos ( θ ) & 0 & b \\ 0 & 0 & 1 & 0 \\ 0 & 0 & 0 & 1\end{matrix} ] .$$

In the BH grid coordinate system, the four corner points of the BH grid are given by:

$$CLT = ( 0 , 0 , 0 )$$

$$CRT = ( {t}_{BH} , 0 , 0 )$$

$$CRB = ( {t}_{BH} , {h}_{BH} , 0 )$$

$$CLB = ( 0 , {t}_{BH} , 0 )$$

The BH grid corner points can be represented as BH_GRID_{BH}=[CLT, CRT, CRB, CLB]. These four corner points can be mapped to the 3D world coordinates using Equation 1 above, and four landmarks BH_GRID that are shape invariant can be obtained.

In another aspect of the principles of the present disclosure—in addition to deriving a BH grid (e.g., for ACL repair) as described above—the four corner points of the derived BH grid can be used advantageously as landmarks for an initial alignment between a statistical shape model (SSM) of the femur being registered, or “fitted”, to the patient's femur. By providing an initial alignment and just a sparse set of 3D point data, a model of the femur useful for computer aided surgery on the patient can be created by the surgeon intraoperatively and without requiring collection of pre-operative images or creation of the model based on such pre-operative images.

Typically, when registering an SSM to a femur, a surgeon first acquires what they believe to be 3D locations of landmarks as inputs for initially registering the SSM itself (which may be referred to as landmark-based initialization). However, landmark-based initialization tends to be inaccurate and may not provide adequate initial alignment. Conversely, the four corners of the BH grid established as described in the present disclosure may be used for a registration procedure instead of the landmark-based initialization. The BH grid corner approach described herein provides more accurate alignment (relative to landmark-based initialization) between the actual femur and the SSM.

As an example, in an intra-operative stage, the surgeon can digitize 3D femur locations (e.g., sparse intra-operative points and/or trajectories, which may be referred to herein as “sparse trajectories”) using an instrumented tool. A 3D femur model can be inferred from these sparse 3D trajectories. This is achieved using an SSM representation.

SSMs can be used to capture the inherent variability and statistical properties of anatomical shapes within a population. SSMs are constructed by first acquiring a large dataset of 3D shapes (e.g., femurs) from a diverse group of individuals. These shapes are then aligned and processed to create a statistical representation of the shape variation within the population. Once the SSM is constructed, the sparse 3D point data is fit to SSM. This can be achieved through a process called shape registration, model fitting, or shape morphing. Shape registration is used to find the SSM instance that best matches the sparse point data in view of the statistical variations captured in the model (i.e., the SSM). In an example, optimization techniques may be used to iteratively adjust the SSM parameters until a best fit is achieved.

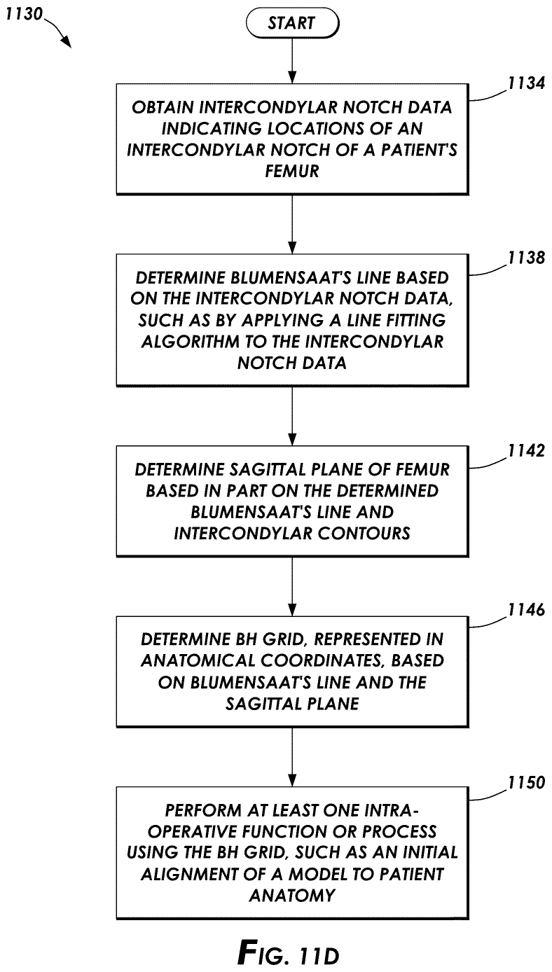

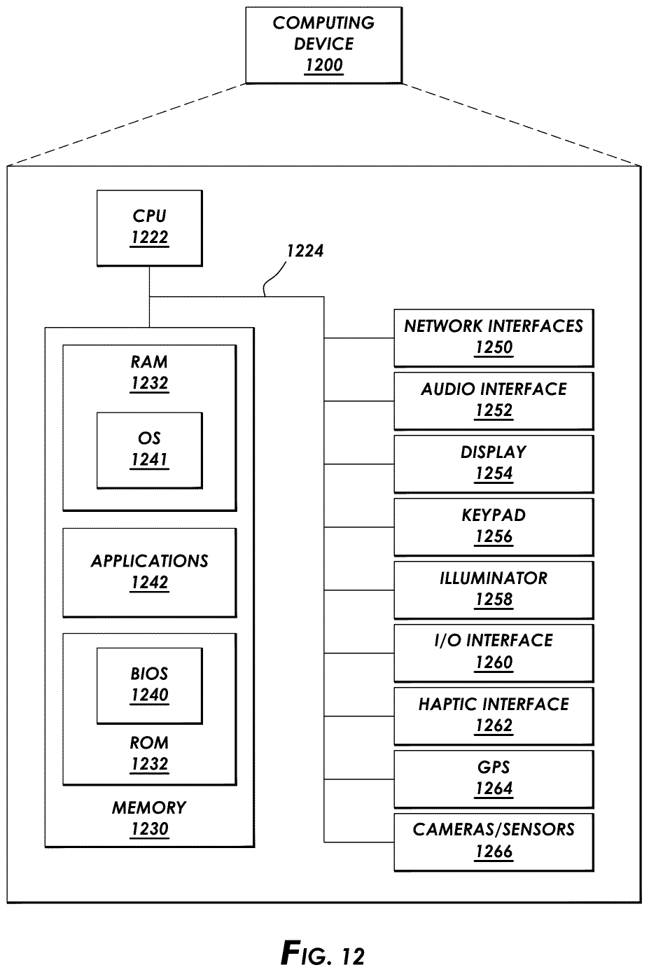

To fit the sparse 3D points to the SSM, an initial alignment between the two 3D data sources is obtained. Typically this is achieved by, in addition to the sparse 3D points, also acquiring (intra-operatively) 3D landmarks whose correspondence is known in the SSM. FIG. 11A illustrates an example 3D bone model 1100 with three example landmarks 1102, 1104, and 1106 acquired on a femur surface. As described above, landmark-based initialization is inaccurate and does not provide an adequate initial alignment for the SSM to work properly.

Conversely, systems and methods of the present disclosure are configured to use the four BH grid corners as described above for performing the initial alignment. In an example, the surgeon, in addition to digitizing the sparse intra-operative data, also digitizes Blumensaat's line as described in FIG. 10A and both intercondylar arcs as described in FIGS. 10C and 10D. From these digitization curves, it is possible to extract the BH grid and the corresponding four corner points, which can be represented as BH_GRID_{REAL}.

For the SSM, Blumensaat's line and both intercondylar arcs are extracted (manually and/or using various automatic techniques), from which the BH grid and its four corner points are estimated using the techniques described above, which can be represented as BH_GRID_{SSM}.

FIGS. 11B and 11C illustrate computation of the sagittal plane and BH grid using the techniques described herein. FIG. 11B corresponds to a model 1112 of a femur from an actual patient and FIG. 11C corresponds to an SSM 1114. An estimated BH grid plane is shown at 1116, with four corner points 1118 corresponding to the corner points of the corresponding BH grid. The four corner points 1118 are used for aligning a patient's anatomy, such as anatomy represented, for illustration only, by the femur model 1112, with the SSM 1114. As described herein, the femur model 1112 is not required to perform the image-free techniques of the present disclosure and is provided in FIG. 11B simply to illustrate the relationship between the sparse data/points, the BH grid plane 1116, and example patient anatomy.

In other words, with the sparse points of intercondylar arches 1120 and 1122 and Blumensaat's line 1124 (e.g., as obtained by the surgeon intra-operatively), the BH grid and the corresponding four corner points, which can be represented as BH_GRID_{REAL}, are extracted. The BH grid and corresponding four corner points can be similarly extracted for the SSM 1114 and represented as BH_GRID_{REAL}. The 3D points corresponding to BH_GRID_{REAL} and BH_GRID_{SSM} can be registered using any 3D registration algorithm or technique, such as a Procrustes method. This registration includes a transformation that is used to align the intra-operatively digitized sparse data shown in the model 1112 with the SSM 1114. This technique as described herein is referred to as BH-based initialization.

Although described above with respect to SSM techniques, other example techniques may be used for fitting sparse points to an anatomical shape model, such as Deep Implicit Shape Models, Deep Atlas models, Neural surface reconstruction, and so on.

In some examples, acquisition of sparse 3D trajectories, Bluemensaat's line, and the intercondylar arcs may be performed using touchless (i.e., non-touch-based) techniques, such as various AI techniques, techniques using depth sensors or structured light sensors, etc.

The four corner points computed from the model 1112 and the SSM 1114 can be registered using any 3D registration algorithm or technique, such as techniques based on computer vision, algebra, artificial intelligence, deep learning, etc.

FIG. 11D shows an example image-free (e.g., without images obtained pre-operatively) Process (or method) 1130 for obtaining, intra-operatively, a BH grid from sparse 3D point/trajectory data. In some examples, the Process 1130 includes and/or is followed by steps for using the four corner points of the BH grid to perform initial alignment between a model of a patient's anatomy (e.g., the sparse 3D data) and an SSM. Steps of the Process 1130 may be performed by one or more processors, computing devices or systems, etc. as described herein, such as one or more computing devices of the system 100, a computing device 1200 as described below, etc.

At 1134, the Process 1130 includes obtaining intercondylar notch data indicating locations of an intercondylar notch of a patient's femur as described herein. For example, the data may correspond to digitized points identified, intra-operatively, by a surgeon, such as via a registration process using an appropriate tool, fiducial markers, etc.

At 1138, the Process 1130 includes determining Blumensaat's line based on the intercondylar notch data. For example, Blumensaat's line may be determined by applying a line fitting algorithm to the intercondylar notch data.

At 1142, the Process 1130 includes determining a sagittal plane based in part on the determined Blumensaat's line. The sagittal plane is a plane that (i) includes Blumensaat's line and is approximately orthogonal to planes T and T′ as described herein. In one example, determining the sagittal plane includes identifying points along medial and lateral intercondylar arcs of the femur. For example, the identified points correspond to intercondylar contours (i.e., of intercondylar arcs) as described herein. In an example, the points of the intercondylar contours may be identified by the surgeon intra-operatively as described herein. Paths of contact of the condyles may be referenced/referred to based on an assumption that the intercondylar contours are parallel to the paths of contact. Accordingly, coordinates of the sagittal plane, which are dependent upon locations/coordinates of the paths of contact, can be calculated based on the points of the intercondylar contours and Blumensaat's line as described above in more detail.

At 1146, the Process 1130 includes computing, storing, etc. a BH grid (e.g., a coordinate representation of the BH grid) based on Blumensaat's line and the sagittal plane. For example, world marker coordinates are mapped to XR plane coordinates, which are then mapped to anatomical coordinates. The anatomical coordinates are related to the BH grid via a transformation process as described above in more detail. Accordingly, the BH grid is represented in anatomical coordinates, including four (4) corner points as described herein.

At 1150, the Process 1130 includes performing at least one intra-operative function or process using the BH grid. In an example, the Process 1130 includes using the BH grid and the four corner points of the BH grid to perform an initial alignment of a model (e.g., an SSM) to patient anatomy as described herein.

FIG. 12 is a block diagram illustrating a computing device 1200 (e.g., UE 106, as discussed above) showing an example of a client device or server device used in the various embodiments of the disclosure. For example, one or more of the computing devices 1200 may be configured to, individually or collectively, perform the functions of the systems and methods of the present disclosure as described in FIGS. 10A-10G and 11A-11D.

The computing device 1200 may include more or fewer components than those shown in FIG. 12, depending on the deployment or usage of the device 1200. For example, a server computing device, such as a rack-mounted server, may not include audio interfaces 1252, displays 1254, keypads 1256, illuminators 1258, haptic interfaces 1262, GPS receivers 1264, or cameras/sensors 1266. Some devices may include additional components not shown, such as GPU devices, cryptographic co-processors, AI accelerators, or other peripheral devices.

As shown in FIG. 12, the device 1200 includes a central processing unit (CPU) 1222 in communication with a mass memory 1230 via a bus 1224. The computing device 1200 also includes one or more network interfaces 1250, an audio interface 1252, a display 1254, a keypad 1256, an illuminator 1258, an input/output interface 1260, a haptic interface 1262, an optional GPS receiver 1264 (and/or an interchangeable or additional GNSS receiver) and a camera(s) or other optical, thermal, or electromagnetic sensors 1266. Device 1200 can include one camera/sensor 1266 or a plurality of cameras/sensors 1266. The positioning of the camera(s)/sensor(s) 1266 on the device 1200 can change per device 1200 model, per device 1200 capabilities, and the like, or some combination thereof.

In some embodiments, the CPU 1222 may comprise a general-purpose CPU. The CPU 1222 may comprise a single-core or multiple-core CPU. The CPU 1222 may comprise a system-on-a-chip (SoC) or a similar embedded system. In some embodiments, a GPU may be used in place of, or in combination with, a CPU 1222. Mass memory 1230 may comprise a dynamic random-access memory (DRAM) device, a static random-access memory device (SRAM), or a Flash (e.g., NAND Flash) memory device. In some embodiments, mass memory 1230 may comprise a combination of such memory types. In one embodiment, the bus 1224 may comprise a Peripheral Component Interconnect Express (PCIe) bus. In some embodiments, the bus 1224 may comprise multiple busses instead of a single bus.

Mass memory 1230 illustrates another example of computer storage media for the storage of information such as computer-readable instructions, data structures, program modules, or other data. Mass memory 1230 stores a basic input/output system (“BIOS”) 1240 for controlling the low-level operation of the computing device 1200. The mass memory also stores an operating system 1241 for controlling the operation of the computing device 1200.

Applications 1242 may include computer-executable instructions which, when executed by the computing device 1200, perform any of the methods (or portions of the methods) described previously in the description of the preceding Figures. In some embodiments, the software or programs implementing the method embodiments can be read from a hard disk drive (not illustrated) and temporarily stored in RAM 1232 by CPU 1222. CPU 1222 may then read the software or data from RAM 1232, process them, and store them to RAM 1232 again.

The computing device 1200 may optionally communicate with a base station (not shown) or directly with another computing device. Network interface 1250 is sometimes known as a transceiver, transceiving device, or network interface card (NIC).

The audio interface 1252 produces and receives audio signals such as the sound of a human voice. For example, the audio interface 1252 may be coupled to a speaker and microphone (not shown) to enable telecommunication with others or generate an audio acknowledgment for some action. Display 1254 may be a liquid crystal display (LCD), gas plasma, light-emitting diode (LED), or any other type of display used with a computing device. Display 1254 may also include a touch-sensitive screen arranged to receive input from an object such as a stylus or a digit from a human hand.

Keypad 1256 may comprise any input device arranged to receive input from a user. Illuminator 1258 may provide a status indication or provide light.

The computing device 1200 also comprises an input/output interface 1260 for communicating with external devices, using communication technologies, such as USB, infrared, Bluetooth™, or the like. The haptic interface 1262 provides tactile feedback to a user of the client device.

The GPS transceiver 1264 can determine the physical coordinates of the computing device 1200 on the surface of the Earth, which typically outputs a location as latitude and longitude values. GPS transceiver 1264 can also employ other geo-positioning mechanisms, including, but not limited to, triangulation, assisted GPS (AGPS), E-OTD, CI, SAI, ETA, BSS, or the like, to further determine the physical location of the computing device 1200 on the surface of the Earth. In one embodiment, however, the computing device 1200 may communicate through other components, provide other information that may be employed to determine a physical location of the device, including, for example, a MAC address, IP address, or the like.

For the purposes of this disclosure a module is a software, hardware, or firmware (or combinations thereof) system, process or functionality, or component thereof, that performs or facilitates the processes, features, and/or functions described herein (with or without human interaction or augmentation). A module can include sub-modules. Software components of a module may be stored on a computer readable medium for execution by a processor. Modules may be integral to one or more servers, or be loaded and executed by one or more servers. One or more modules may be grouped into an engine or an application.

Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible.

Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

What is claimed is:

1. A method, comprising:
receiving, by a computing device, a plurality of identified points corresponding to an intercondylar notch of a femur of a patient, a medial intercondylar arc of the femur, and a lateral intercondylar arc of the femur;
determining, by the computing device and based on the plurality of points, a Blumensaat line, a medial contour of the medial intercondylar arc, and a lateral contour of the lateral intercondylar arc;
determining, by the computing device, a sagittal plane for the femur based on the Blumensaat line, the medial contour of the medial intercondylar arc, and the lateral contour of the lateral intercondylar arc; and
performing at least one function of a surgical procedure associated with the femur based on the determined sagittal plane.

2. The method of claim 1, wherein:
the plurality of points includes:
a first set of 3D points along a roof of the intercondylar notch;
a second set of 3D points along the medial intercondylar arc; and
a third set of 3D points along the lateral intercondylar arc;
determining, by the computing device, the Blumensaat line based on the first set of 3D points;
determining, by the computing device, the medial contour based on the second set of 3D points;
determining, by the computing device, the lateral contour based on the third set of 3D points;
analyzing, by the computing device, the Blumensaat line, the medial contour, and the lateral contour, and determining a pair of 3D points, each point of the pair of 3D points being on a respective one of the medial contour and the lateral contour, having parallel tangent planes and having a vector between the pair of 3D points that is orthogonal to the Blumensaat line; and
determining, by the computing device, the sagittal plane for the femur based on the vector and the Blumensaat line.

3. The method of claim 1, further comprising obtaining at least a portion of the plurality of points intra-operatively.

4. The method of claim 1, wherein the determining of the Blumensaat line, the medial contour, and the lateral contour are performed without using a pre-operative image of the femur.

5. The method of claim 1, further comprising:
determining, by the computing device, a Bernard-Hertel (BH) grid corresponding to the femur based on the Blumensaat line and the sagittal plane.

6. The method of claim 5, further comprising:
aligning, by the computing device, a statistical shape model (SSM) to the femur based on locations of four corners of the BH grid.

7. The method of claim 6, wherein aligning the SSM includes aligning a BH grid of the SSM with the BH grid corresponding to the femur.

8. A non-transitory computer-readable storage medium tangibly encoded with computer-executable instructions, that when executed by at least one processor, perform a method comprising:
receiving a plurality of identified points corresponding to an intercondylar notch of a femur of a patient, a medial intercondylar arc of the femur, and a lateral intercondylar arc of the femur;
determining, based on the plurality of points, a Blumensaat line, a medial contour of the medial intercondylar arc, and a lateral contour of the lateral intercondylar arc;
determining a sagittal plane for the femur based on the Blumensaat line, the medial contour of the medial intercondylar arc, and the lateral contour of the lateral intercondylar arc; and
performing at least one function of a surgical procedure associated with the femur based on the determined sagittal plane.

9. The computer-readable storage medium of claim 8, wherein:
the plurality of points includes:
a first set of 3D points along a roof of the intercondylar notch;
a second set of 3D points along the medial intercondylar arc; and
a third set of 3D points along the lateral intercondylar arc;
and wherein the method further comprises:
determining the Blumensaat line based on the first set of 3D points;
determining the medial contour based on the second set of 3D points;
determining the lateral contour based on the third set of 3D points;
analyzing the Blumensaat line, the medial contour, and the lateral contour, and determining a pair of 3D points, each point of the pair of 3D points being on a respective one of the medial contour and the lateral contour, having parallel tangent planes and having a vector between the pair of 3D points that is orthogonal to the Blumensaat line; and
determining the sagittal plane for the femur based on the vector and the Blumensaat line.

10. The computer-readable storage medium of claim 8, wherein the method further includes obtaining at least a portion of the plurality of points intra-operatively.

11. The computer-readable storage medium of claim 8, wherein the determining of the Blumensaat line, the medial contour, and the lateral contour are performed without using a pre-operative image of the femur.

12. The computer-readable storage medium of claim 8, wherein the method further comprises:
determining a Bernard-Hertel (BH) grid corresponding to the femur based on the Blumensaat line and the sagittal plane.

13. The computer-readable storage medium of claim 12, wherein the method further comprises:
aligning a statistical shape model (SSM) to the femur based on locations of four corners of the BH grid.

14. The computer-readable storage medium of claim 13, wherein aligning the SSM includes aligning a BH grid of the SSM with the BH grid corresponding to the femur.

15. A system, comprising:
one or more processors configured to:
receive a plurality of identified points corresponding to an intercondylar notch of a femur of a patient, a medial intercondylar arc of the femur, and a lateral intercondylar arc of the femur;
determine, based on the plurality of points, a Blumensaat line, a medial contour of the medial intercondylar arc, and a lateral contour of the lateral intercondylar arc;
determine a sagittal plane for the femur based on the Blumensaat line, the medial contour of the medial intercondylar arc, and the lateral contour of the lateral intercondylar arc; and
perform at least one function of a surgical procedure associated with the femur based on the determined sagittal plane.

16. The system of claim 15, wherein:
the plurality of points includes:
a first set of 3D points along a roof of the intercondylar notch;
a second set of 3D points along the medial intercondylar arc; and
a third set of 3D points along the lateral intercondylar arc;
and wherein the one or more processors are further configured to:
determine the Blumensaat line based on the first set of 3D points;
determine the medial contour based on the second set of 3D points;
determine the lateral contour based on the third set of 3D points;
analyze the Blumensaat line, the medial contour, and the lateral contour, and determine a pair of 3D points, each point of the pair of 3D points being on a respective one of the medial contour and the lateral contour, having parallel tangent planes and having a vector between the pair of 3D points that is orthogonal to the Blumensaat line; and
determine the sagittal plane for the femur based on the vector and the Blumensaat line.

17. The system of claim 15, wherein the one or more processors are configured to receive at least a portion of the plurality of points during an intra-operative procedure.

18. The system of claim 15, wherein the determining of the Blumensaat line, the medial contour, and the lateral contour are performed without using a pre-operative image of the femur.

19. The system of claim 15, wherein the one or more processors are configured to determine a Bernard-Hertel (BH) grid corresponding to the femur based on the Blumensaat line and the sagittal plane.

20. The system of claim 19, wherein the one or more processors are configured to align a statistical shape model (SSM) to the femur based on locations of four corners of the BH grid.
